(19) United States
(12) Patent Application Publication
(10) Pub. No.: US 2018/0118598 A1
(43) Pub. Date: May 3, 2018

(54) NITRATE REMOVAL BY ION EXCHANGE AND BIOREGENERATION

(71) Applicant: TECHNION RESEARCH & DEVELOPMENT FOUNDATION LIMITED, Haifa (IL)

(72) Inventors: Michal GREEN, Haifa (IL); Sheldon TARRE, Kibbutz Yagur (IL)

(21) Appl. No.: 15/571,926

(22) PCT Filed: May 3, 2016
(86) PCT No.: PCT/IL2016/050459

§ 371 Date: Nov. 6, 2017

(30) Foreign Application Priority Data
May 7, 2015 (GB) 1507823.1

(51) Int. Cl. C02F 9/00; B01J 41/20; B01J 47/02; B01J 49/75; B01J 49/57; A61L 2/18; A61L 2/26; B01D 15/36; B01D 15/18; B01D 15/20

(52) U.S. Cl. CPC C02F 9/00; B01J 41/20; B01J 47/02; B01J 49/75; B01J 49/57; C02F 1/42; A61L 2/26; B01D 15/361; B01D 15/1864; B01D 15/203; A61L 2/186

(57) ABSTRACT

A system for nitrate removal from water combining: an ion exchange unit comprising at least one column of an ion exchange resin, a brine bioregeneration circuit comprising a sequential batch reactor (SBR), and an ozonation unit, is disclosed. A method for nitrate removal from water is further disclosed.

This application claims priority from G.B. Patent Application No. GB1507823.1, filed on May 7, 2015. The content of the above document is incorporated by reference as if fully set forth herein.

### FIELD OF INVENTION

The invention relates to the field of water treatment, and more specifically, but not exclusively to removal of nitrate from water.

### BACKGROUND OF THE INVENTION

Excessive nitrate concentration is a major cause of closing potable water wells throughout the world. Treatment options of nitrate bearing waters involve nitrate separation and/or reduction to N₂ (Seidal et al. An Assessment of the State of Nitrate Treatment Alternatives, Final Report, The American Water Works Association. 136 p. 2011).

Separation is the most common strategy and includes technologies such as reverse osmosis (RO), ion exchange (IX) and electrodialysis (ED). These technologies are cost effective, reliable and safe. However, they become impractical in locations where brine disposal is either too expensive or restricted, particularly at inland sites.

The process currently most used for nitrate removal from ground water is the ion exchange process; specifically—anion exchange. Since this process does not destroy the nitrate, it eventuates in a more concentrated form in the waste streams. Since these waste streams inherently comprise considerable amounts of salt, disposal of nitrate-contaminated brine has become a relevant environmental issue.

The other employed option is direct biological denitrification, heterotrophic or autotrophic where nitrate is transformed into harmless nitrogen gas and no brine is produced. However, application of this technology requires extensive post treatment due to health concerns associated with exposure of drinking water to bacteria, nitrite and residual organics. In many places, the low acceptance of biologically treated drinking water by the regulators limits the application of these technologies. Catalytic non-biotic nitrate reduction using metals or hydrogen has also been suggested as a brine free nitrate removal strategy. However, such methods may release nitrite, ammonia and toxic metal catalysts to the product water and have not been demonstrated yet at full scale.

Several alternative strategies attempt to combine physico-chemical technologies with biological technologies in order to avoid the downsides of each separate technology. In one approach, nitrate is firstly removed from the feed water using a nitrate-selective ion exchange resin and subsequently regenerated using a brine solution in a closed loop fashion. The nitrate-loaded regenerant is treated for reuse by biological denitrification. As compared with conventional IX regeneration it is possible to reach a significant reduction in waste volume and in regeneration salt requirement.

Under prolonged operation, dissolved organic carbon (DOC) concentrations in the recycled brine can easily accumulate to 300-400 mg/L (McAdam et al., Water Research 44 (1), 69-76. 2010). These organics can lead to IX resin fouling, reduced IX capacity (Bae et al., Water Research 36 (13), 3330-3340, 2002) and treated water bacterial contamination due to bacterial growth on the resin (van der Hoek et al., Wasser Abwass. Forsch., 20, 155-1601987).

Intermittent replacement of the DOC contaminated regenerant increases salt demand and the amount of waste brine requiring disposal. In addition, combined ion exchange bioregeneration research-level systems have shown relatively high amounts of chloride addition to the treated water, greater than the stoichiometric amount of nitrate removed due to sulfate ion exchange and because of possible insufficient rinsing of the resin with freshwater after the regeneration step.

### SUMMARY OF THE INVENTION

The invention relates to the field of water treatment, and more specifically, but not exclusively, to removal of nitrate from water.

According to an aspect of some embodiments of the present invention, there is provided a method of removing nitrates from contaminated water, the method comprising the steps of (“service steps”): contacting the nitrate contaminated water with one or more columns of ion exchange resins having an affinity to nitrate, thereby removing nitrate from the water and forming a product water having reduced nitrate content and loading nitrate in the one or more columns of the exchange resins, and separating the reduced nitrate content product water from the nitrate loaded columns of the ion exchange resin.

According to some embodiments, the method further comprises the steps of (“regeneration step”): contacting the nitrate loaded columns of the ion exchange resin with a fed brine solution having nitrate desorbing content thereby forming a regenerated ion exchange resin having reduced nitrate load, and removing the brine solution from the treated ion exchange resin.

According to some embodiments, the method further comprises the regeneration steps of: contacting the brine solution to sequential batch reactor (SBR) comprising denitrifying bacteria, adding an electron donor to the SBR thereby essentially removing nitrate from the brine solution, performing sedimentation of the brine solution and adding salt thereto to thereby remove excess denitrifying bacterial biomass therefrom, contacting the brine solution with O₃ thereby disinfecting and/or removing remaining suspended solids, turbidity and dissolved organic-based component in the brine.

According to some embodiments, the desorbing content comprises chloride anions in concentration of at least 10,000 mg/L. According to some embodiments, the dissolved organic-based component is, or derived from, denitrifying biomass and/or bacterial component.

According to some embodiments, the method further comprises keeping a pH of the sequential batch reactor (SBR) at a value that ranges from about pH 7 to about 9, e.g., by acid addition.

According to some embodiments, the method further comprises using oxidation reduction potential (ORP) measurement. In some embodiments the measurement is of the sequential batch reactor so as to control electron donor addition. In some embodiments the measurement is of ozonation unit so as to control ozone addition and flow of brine from ozonation unit to ion exchange unit.

According to some embodiments, controlling the electron donor addition is performed in aliquots at a specified time intervals so as to allow minimizing electron donor addition and dissolved organic-based component.

According to some embodiments, the method further comprises a step of discharging the brine in the sequential batch reactor by ORP control.

According to some embodiments, the steps of contacting the nitrate contaminated water with the columns of ion exchange resins, and the step of separating the reduced nitrate content product water from the nitrate loaded columns are performed repeatedly.

According to some embodiments, the steps of contacting the nitrate loaded columns of the ion exchange resin with fed brine up to the step of contacting the brine solution with O₃ are performed repeatedly.

According to some embodiments, the steps of contacting the nitrate loaded columns of the ion exchange resin with a fed brine up to the step of contacting the brine solution with O₃ are recycled.

According to some embodiments, the steps of contacting the nitrate contaminated water with the columns of ion exchange resins, and the step of separating the reduced nitrate content product water from the nitrate loaded columns are recycled. According to some embodiments, the method is performed such that at least 75% (wt.) of the brine solution is recycled.

According to some embodiments, one or more of the service steps and one or more of generation steps are performed simultaneously.

According to some embodiments, one or more of the service steps and one or more of generation steps are performed simultaneously in a different column of ion exchange resin.

According to an aspect of some embodiments of the present invention, there is provided a system comprising an ion exchange unit comprising at least one column of an ion exchange resin, a brine bioregeneration circuit comprising a sequential batch reactor (SBR), and an ozonation unit, wherein the, ion exchange unit, SBR, and ozonation unit are in fluid communication to each other. According to some embodiments, the ion exchange resin is a nitrate selective resin. According to some embodiments, the fluid is a brine solution. According to some embodiments, the brine solution comprises chloride anions in concentration that ranges from about 10,000 mg/L to about 50,000 mg/L. According to some embodiments, the system of described herein, further comprises a pH meter for determining a pH of a fluid inside the sequential batch reactor.

According to some embodiments, the ion exchange unit comprises at least two columns of an ion exchange resin.

According to some embodiments, the system further comprises a pipe attached to, or integrally formed with the SBR, wherein the pipe is configured to lead an electron donor into the sequential batch reactor. According to some embodiments, the electron donor is one or more materials selected from the group consisting of: acetic acid, ethanol, and hydrogen gas.

According to some embodiments, the system further comprises a pipe attached to, or integrally formed with the SBR, wherein the pipe is configured to lead an acid into the SBR. According to some embodiments, the acid is a hydrochloride acid.

According to some embodiments, SBR further comprises denitrifying bacteria.

According to some embodiments, the system further comprises a pipe attached to, or integrally formed with the SBR or the settling tank, wherein the pipe is configured to lead salt solution into the SBR or the settling tank, the salt solution comprising chloride anions. According to some embodiments, the salt is sodium chloride.

According to some embodiments, the nitrate-reduced water comprises nitrate in concentration of less than 15 mg N/L. According to some embodiments, the nitrate-reduced water comprises chloride anions in concentration of less than 430 mg/L.

According to some embodiments, the system further comprises a settling tank, being in fluid communication to the ozonation unit and to the SBR. According to some embodiments, the system further comprises a pipe attached to, or integrally formed with the ozonation unit, wherein the pipe is configured to lead the brine solution out of the ozonation unit and enters the ion exchange unit. According to some embodiments, the system further comprises a recirculation brine pump in the brine bioregeneration circuit, the pump being configured to transfer the brine solution to ion exchange unit and fluidize the ion exchange resin. According to some embodiments, the system further comprises a pipe attached to, or integrally formed with the ion exchange unit, wherein the pipe is configured to lead a disinfectant solution to the ion exchange unit. According to some embodiments, the disinfectant solution is hydrogen peroxide.

According to some embodiments, the SBR of the brine bioregeneration circuit is disposed downstream of the ion exchange unit and is configured to receive the brine solution from at least one ion exchange column.

According to some embodiments, the settling tank is disposed downstream of the SBR and is configured to receive the fluid from the sequential batch reactor.

According to some embodiments, the ozonation unit is disposed downstream of the settling tank and is configured to receive the fluid from the settling tank.

According to some embodiments, the ion exchange unit comprises a first water inlet configured to provide nitrate contaminated water to the at least one column of the ion exchange resin, a second water inlet configured to provide a brine solution from the ozonation unit to the at least one column of the ion exchange resin, a first water outlet configured to allow an exit of nitrate-reduced water from the least one ion column of the ion exchange resin, and a second water outlet configured to transfer the brine solution from the at least one column of the ion exchange resin to the sequential batch reactor.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods, systems, and/or materials are described below. In case of conflict, the patent specification, including definitions, will control. In addition, the materials, methods, and examples are illustrative only and are not intended to be necessarily limiting.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIGS. 1A-C show schematic illustrations of the filtration system in a flow sheet of an exemplary filtration system (block diagram; FIG. 1A), and in a close-up view of the Ion Exchange unit (FIG. 1B), and the Brine Biogeneration circuit (FIG. 1C).

FIG. 2 presents graphs presenting typical concentrations of NO₃⁻ (open triangle) and NO₂ (closed circle) and oxidation reduction potential (ORP; open diamond) during the sequential batch reactor (SBR) denitrification. Arrows show the times of stepwise addition of ethanol and % of the total ethanol dose given.

FIG. 3 presents graphs showing SBR denitrification rate at the beginning of the batch: initial 60 minute period without ethanol dosing had rate of 2.0 g N-N/hr (closed diamonds with solid line, r=0.99), and after ethanol addition at 90 minutes (marked with an arrow), the rate increased to 8.5 g N/hr (open circle, r=0.99).

### DETAILED DESCRIPTION OF THE INVENTION

The present invention, in some embodiments thereof, relates to a method of ion exchange (IX) and brine bio-regeneration and systems capable of same.

Before explaining at least one embodiment of the invention in detail, it is to be understood that the invention is not necessarily limited in its application to the details set forth in the following description or exemplified by the Examples.

The invention is capable of other embodiments or of being practiced or carried out in various ways. The system of the kind provided herein may comprise units with various water treatment functions. The choice of treatment functions to be included may be made based on the specific properties and quality of the water to be treated, on the basis of intended properties of the filtered water, based on regulatory requirements and many others. As will be appreciated, the system provided herein is not limited to a certain combination of water filtration units.

According to one aspect of the present invention there is provided a system for removing nitrates from water. The system may comprise an ion exchange unit comprising at least one column of an ion exchange resin, a brine bioregeneration circuit comprising a sequential batch reactor (SBR), and a disinfection unit, wherein the ion exchange unit, the SBR, and the disinfection unit are in fluid communication to each other, and the fluid is e.g., a brine solution. In some embodiments, the SBR is disposed downstream of the ion exchange unit and is configured to receive the brine solution from at least one ion exchange column. In some embodiments, the disinfection unit is disposed downstream of the SBR.

In some embodiments, by “at least one column” it is meant e.g., 1 column, at least 2 columns, at least 3 columns, at least 4 columns, at least 5 columns, at least 6 columns, at least 7 columns, at least 8 columns, at least 9 columns, or at least 10 columns.

In some embodiments, the system further comprises a settling tank, being in fluid communication with the SBR, and the disinfection unit.

In some embodiments the disinfection unit is an ozonation unit.

In some embodiments, the system allows a process of removing nitrate from the water and forming a product water having reduced nitrate content while minimizing the chloride addition during the process.

In some embodiments, the chloride concentration in the brine is maintained at e.g., 5 to 10 g/L, 10 to 15 g/L, or 15 to 20 g/L. In exemplary embodiments, the chloride concentration in the brine is maintained at 14-16 g/L.

Accordingly, in some embodiments of the present invention, the disclosed system offers two modes of operation: a) removing nitrates from contaminated water by an ion exchange resin; and b) brine bioregeneration circuit in which the ion exchange resin is regenerated, and the nitrate is reduced therefrom.

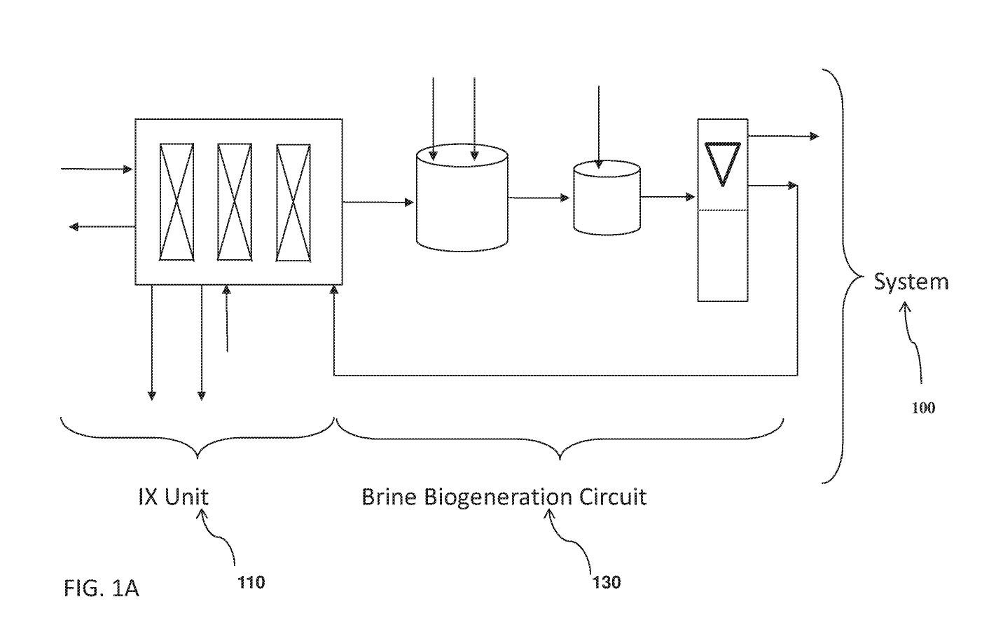

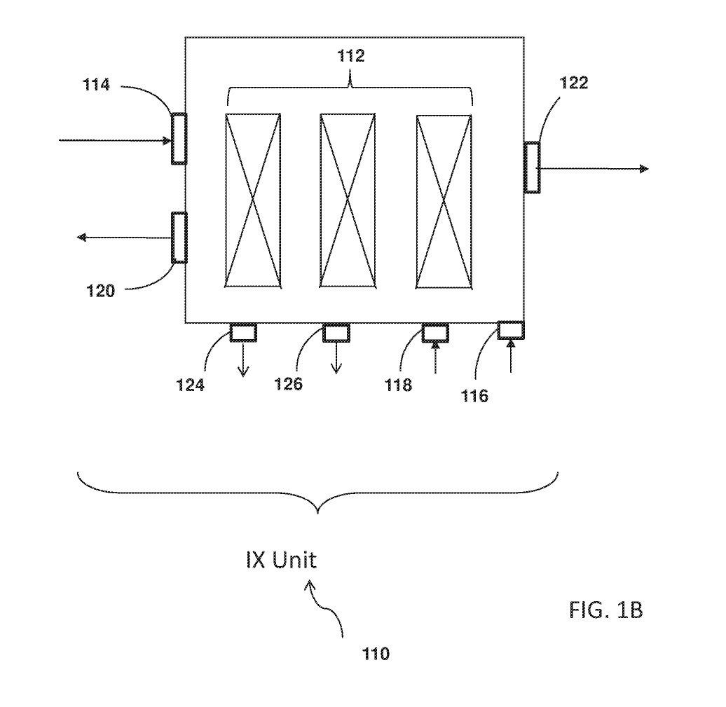

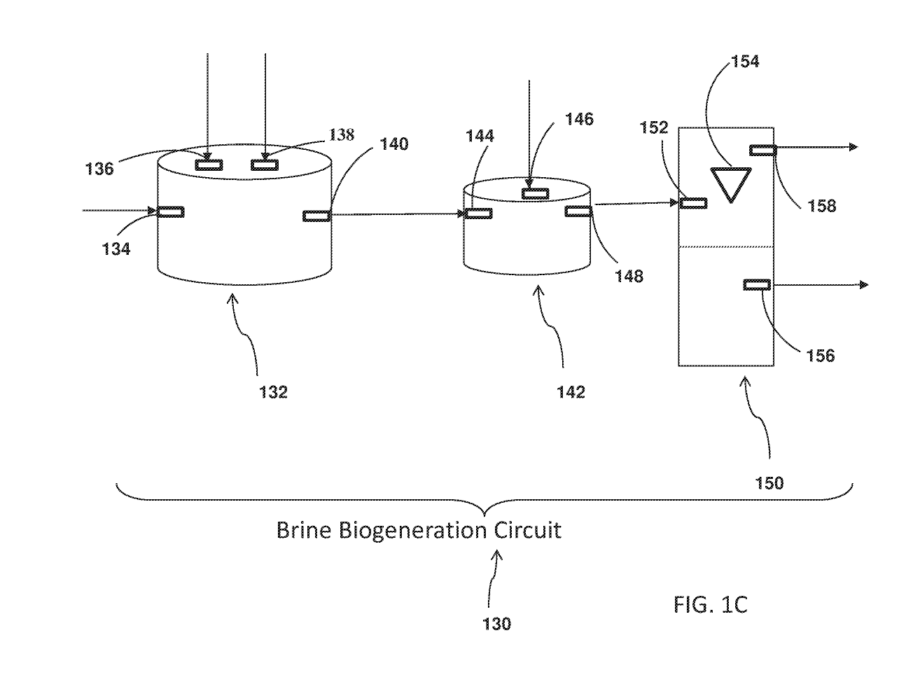

Reference is now made to FIGS. 1A-C, which, taken together, schematically illustrate a configuration of an exemplary system according to an embodiment of the present invention. FIG. 1A shows a schematic illustration of an exemplary filtration system in a flow sheet.

The system 100 may have a housing 110. Housing 110 may be made of a rigid, durable material, such as, without limitation, aluminum, stainless steel, a hard polymer and/or the like.

FIG. 1B presents a detailed close-up view of housing (also referred to as “IX Unit”) 110. Housing 110 may have a cylindrical, conical, rectangular or any other suitable shape. Housing 110 may prevent unwanted foreign elements from entering thereto. Housing 110 may comprise one or more (e.g., 1, 2, 3, 4, 5, 6, 7, 8, 9, or 10) columns 112 as described hereinthroughout, configured to allow water and/or brine solution to pass therethrough.

Housing 110 may have a water inlet port 114. Water inlet port 114 may include a pipe of various shapes and sizes, connected to, attached to or integrally formed with the housing 110. Water inlet port 114 may allow unfiltered water to enter housing 110.

Housing 110 may have a brine inlet port 116. Brine inlet port 116 may include a pipe of various shapes and sizes, connected to, attached to or integrally formed with the housing 110. Brine inlet port 116 may allow brine to enter housing 110.

The term “port” as used hereinthroughout, refers to a path for distributing liquid or gas, either on or above ground surface or underground, which may include, without being limited thereto, ducts, pipes, channels, tubes, troughs or other means for distribution. As used herein, the pipe may be adjacent or abutting to housing 110. The Pipe may be a funnel.

Housing 110 may have a disinfectant (e.g., H₂O₂) inlet port 118. Disinfectant inlet port 118 may include a pipe of various shapes and sizes, connected to, attached to or integrally formed with the housing 110. Disinfectant inlet port 118 may allow disinfectant to enter housing 110.

Housing 110 may have water outlet port 120. Water outlet port 120 may be a pipe. Water outlet port 120 may be an opening of various shapes and sizes in housing 110. Water outlet port 120 may be configured as a siphon. Water outlet port 120 may allow filtered water to exit housing 110.

Housing 110 may have brine outlet port 122. Brine outlet port 122 may be a pipe. Brine outlet port 122 may be an opening of various shapes and sizes in housing 110. Brine outlet port 122 may be configured as a siphon. Brine outlet port 122 may allow brine to exit housing system 110 and to flow to the Brine Biogeneration Circuit.

Housing 110 may have brine rinse outlet port 124. Brine rinse outlet port 124 may be a pipe. Brine rinse outlet port 124 may be an opening of various shapes and sizes in housing 110. Brine rinse outlet port 124 may be configured as a siphon. Brine rinse outlet port 124 may allow residual brine to exit housing system 110.

Housing 110 may have fresh water rinse outlet port 126. Fresh water rinse outlet port 124 may be a pipe. Fresh water rinse outlet port 126 may be an opening of various shapes and sizes in housing 110. Fresh water rinse outlet port 126 may be configured as a siphon. Fresh water rinse outlet port 126 may allow residual freshwater (e.g., having a reduced nitrate content) to exit housing system 110.

System 100 may include Brine Biogeneration Circuit (BBC) 130. FIG. 1C presents a detailed close-up view of BBC 130. BBC 130 may have a Sequential Batch Reactor (SBR) 132. SBR 132 may comprise a brine solution. SBR 132 may have a brine inlet port 134. Brine inlet port 134 may include a pipe of various shapes and sizes, connected to, attached to or integrally formed with SBR 132. Brine inlet port 134 may allow brine exiting from housing 110 to enter SBR 132.

SBR 132 may have an electron donor inlet port 136. Electron donor inlet port 136 may include a pipe of various shapes and sizes, connected to, attached to or integrally formed with the SBR 132. Electron donor inlet port 136 may allow a solution comprising electron donor to enter SBR 132.

SBR 132 may have an acid inlet port 138. Acid inlet port 138 may include a pipe of various shapes and sizes, connected to, attached to or integrally formed with SBR 132. Acid inlet port 138 may allow a solution comprising acid to enter SBR 132.

SBR 132 may have brine outlet port 140. Brine outlet port 140 may be a pipe. Brine outlet port 140 may be an opening of various shapes and sizes in SBR 132. Brine outlet port 140 may allow brine to exit SBR 132.

BBC 130 may include pH-meter e.g., for the determining the pH of a fluid inside SBR 132.

BBC 130 may include ORP-meter for the determining the ORP value of the brine solution.

BBC 130 may include settling tank 142. Settling tank 142 may have a brine inlet port 144. Brine inlet port 144 may include a pipe of various shapes and sizes, connected to, attached to or integrally formed with the settling tank 142. Brine inlet port 144 may allow brine exiting from SBR 132 to enter settling tank 142. Settling tank 142 may allow, inter alia, collecting salt and settling sludge that developed in the SBR.

Settling tank 142 may include a salt solution inlet port 146. Salt solution inlet port 146 may include a pipe of various shapes and sizes, connected to, attached to or integrally formed with the settling tank 142. Salt solution inlet port 146 may allow a solution comprising salt (e.g., NaCl) to enter settling tank 142. Settling tank 142 may have salt solution outlet port 148. Salt solution outlet port 148 may be a pipe. Salt solution outlet port 148 may be an opening of various shapes and sizes in settling tank 142. Salt solution outlet port 148 may allow brine to exit settling tank 142 and to enter e.g., the ozonation unit as described below.

BBC 130 may include ozonation unit 150. The term “ozonation unit” refers to a unit in which ozonation, as described hereinthroughout, takes place. As used herein, the term “unit” may refer to an area including one or more equipment items and/or one or more sub-zones. Equipment items can include one or more reactors or reactor vessels, pipes, pumps, oxygen and ozone generators, and/or ORP controller. Additionally, an equipment item, such as a reactor, dryer, or vessel, can further include one or more zones or sub-zones.

Ozonation unit 150 may have a brine inlet port 152. Brine inlet port 152 may include a pipe of various shapes and sizes, connected to, attached to or integrally formed with the ozonation unit 150. Brine inlet port 152 may allow brine exiting from settling tank 142 to enter ozonation unit 150.

Settling tank 142 may be absent such that brine solution may allow to enter ozonation unit 150 from SBR 132.

Ozonation unit 150 may have an ozone inlet port 154. Ozone inlet port 154 may include a pipe of various shapes and sizes, connected to, attached to or integrally formed with ozonation unit 150. Ozone inlet port 154 may allow ozone to enter ozonation unit 150.

Ozonation unit 150 may have a brine outlet port 156. Brine outlet port 156 may be a pipe. Brine outlet port 156 may be an opening of various shapes and sizes in ozonation unit 150. Brine outlet port 156 may allow brine to exit ozonation unit 150 and to enter housing 110 at the brine inner port 116.

Ozonation unit 150 may have foam outlet port 158. Foam outlet port 158 may be a pipe. Foam outlet port 158 may be an opening of various shapes and sizes in ozonation unit 150. Foam outlet port 158 may allow foam generated in ozonation unit 150 to evaporate therefrom. As used herein and in the art, the terms “foam” refers to a three-dimensional porous material having a reticulated configuration in cross section and which is pliable.

The dimensions of each component of the system are selected to be sufficient, for a given desired fluidization and to provide sufficient contact time to provide e.g., a desired level of water consumption and/or brine regeneration.

Conditions may be monitored using any suitable type monitoring devices e.g., a computer-implemented system. Variables that may be tracked include, without limitation, pH, temperature, conductivity, turbidity, dissolved nitrate concentration, oxidation reduction potential (ORP), dissolved oxygen, as well as the concentrations of nitrate and chloride. These variables may be recorded throughout system 100.

A monitoring device, a control unit, or a controller (e.g., computer) may also be used to monitor, control and/or automate the operation of the various components of the systems disclosed herein, such as any of the valves, sensors, weirs, blowers, fans, dampers, pumps, etc.

The present invention may be a system, a method, and/or a computer program product. The computer program product may comprise a computer-readable storage medium. The computer-readable storage medium may have program code embodied therewith. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The program code may be excusable by a hardware processor. The program code may be excusable by a hardware processor to any step of the method, or any part of the system as described hereinbelow.

For example, the program code may be executable by a hardware processor to receive one or more system parameters as input signals, and process the parameters to control the performance of one or more of the following steps:

feeding nitrate contaminated water with one or more columns of ion exchange resins;

controlling the ports (inlets and outlets) of the system as described hereinthroughout;

adding an electron donor as described hereinthroughout;

controlling the denitrification sequential batch reactor (SBR);

controlling the ozonation unit e.g., the amount of the ozone used to reduce the turbidity of the regenerant;

controlling flow configuration of the method steps as described hereinthroughout; and/or controlling the oxidation-reduction potential (ORP).

In some embodiments of the invention, system 100 is outfitted with a pump, e.g., feeding pump and/or a recirculation pump so as to further fluidize the water or the brine. According to an aspect of some embodiments of the present invention, there is provided a method for removing nitrates from contaminated water, using the system described herein.

Hereinthroughout, pump may be electronically controlled, or mechanically controlled.

In some embodiments the method comprises the steps of (also referred to as “service cycle”):

contacting the nitrate contaminated water with one or more columns of ion exchange resins having an affinity to nitrate, thereby removing nitrate from the water and forming a product water having reduced nitrate content and loading nitrate in the one or more columns of the exchange resins; and

separating the reduced nitrate content product water from the nitrate loaded columns of the ion exchange resin.

The method may further comprise forming a regenerated ion exchange resin (also referred to as: “Brine Biogeneration Circuit” in the disclosed system).

Forming a regenerated ion exchange resin may comprise one or more of the steps of (referred to as: “regeneration cycle”):

contacting the nitrate loaded columns of the ion exchange resin with a fed brine solution having nitrate desorbing content thereby desorbing nitrate from the resin and forming a regenerated ion exchange resin having reduced nitrate load; and

removing the brine solution from the treated ion exchange resin;

The method may further comprise one or more of the steps of (in the regeneration cycle):

contacting the brine solution to SBR comprising denitrifying bacteria;

adding an electron donor to the SBR thereby essentially removing nitrate from the brine solution;

performing sedimentation of the brine solution and adding salt thereto to thereby remove excess denitrifying bacterial biomass therefrom; and

contacting the brine solution with O₃ thereby disinfecting and/or removing remaining suspended solids, turbidity and dissolved organic-based component in the brine.

In some embodiments, one or more steps may be performed repeatedly.

In some embodiments, only some of the above-mentioned steps are performed.

In some embodiments, a certain step may be recycled to another step.

In some embodiments, two or more steps are performed at the same time (i.e. simultaneously) via two or more different columns.

In some embodiments, two or more steps are performed simultaneously, wherein at least one step belongs to the in a service cycle, and at least one step belongs to the regeneration cycle.

In some embodiments, the service length is operated at e.g., 1 bed volumes (BV), 10 BV, 20 BV, 30 BV, 40 BV, 50 BV, 60 BV, 70 BV, 80 BV, 90 BV, 100 BV, 110 BV, 120 BV, 130 BV, 140 BV, 150 BV, 160 BV, 170 BV, 180 BV, 190 BV, 200 BV, 210 BV, 220 BV, 230 BV, 240 BV, 250 BV, 260 BV, 270 BV, 280 BV, 290 BV, 300 BV, 310 BV, 320 BV, 330 BV, 340 BV, 350 BV, 360 BV, 370 BV, 380 BV, 390 BV, 400 BV, 410 BV, 420 BV, 430 BV, 440 BV, 450 BV, 460 BV, 470 BV, 480 BV, 490 BV, 500 BV, 510 BV, 520 BV, 530 BV, 540 BV, 550 BV, 560 BV, 570 BV, 580 BV, 590 BV, or 600 BV, including any value and range therebetween.

In some embodiments, the service length is operated at 350 BV to 450 BV. In some embodiments, the service length is operated at 360 BV to 390 BV.

In some embodiments, the service length is operated at e.g., 1 bed volumes (BV), 2 BV, 3 BV, 4 BV, 5 BV, 6 BV, 7 BV, 0 BV, 9 BV, 10 BV, 11 BV, 12 BV, 13 BV, 14 BV, 15 BV, 16 BV, 17 BV, 18 BV, 19 BV, 20 BV, 21 BV, 22 BV, 23 BV, 24 BV, 25 BV, 26 BV, 27 BV, 28 BV, 29 BV, 30 BV, 31 BV, 32 BV, 33 BV, 34 BV, 35 BV, 36 BV, 37 BV, 38 BV, 39 BV, or 40 BV, including any value and range therebetween.

In some embodiments, the term “bed volume” refers to volume per hours of liquid to be treated divided by the volume of resin.

As used herein, the term “repeatedly” designates an action, step or operation that is carried out a number of times or is performed from time-to-time. The term “repeatedly” thus is not intended to imply or require that the step(s) or operation be performed at fixed intervals.

As used hereinthroughout, the terms “fluid communication” or “hydraulically connection” which are used hereinthroughout interchangeably, means fluidically interconnected, and refers to the existence of a continuous coherent flow path from one of the components of the system to the other if there is, or can be established, liquid and/or gas flow through and between the ports even if there exists a valve between the two conduits that can be closed, when desired, to impede fluid flow therebetween. The term “port” refers to a path for distributing liquid or gas, either on or above ground surface or underground, which may include but is not limited to one or more ducts, pipes, channels, tubes, troughs or other means for distribution. Likewise, as may be seen, the terms “upstream” and “downstream” are referred to the direction of flow of the fluid.

In some embodiments, the fluid is a brine solution.

As used herein, the term “brine”, or “brine solution”, is meant to refer to any water-based fluid containing a measurable concentration of an inorganic salt capable to desorb nitrate from the ion exchange resin. The salinity of the brine may range from between e.g., about 5 to about 50 ppt (parts per thousand) which is about 0.5 to 5% salt.

Hereinthroughout, the brine solution, following one cycle, i.e. upon exiting the ion exchange resin, is also referred to as “regenerant”.

Typically, the brine solution exiting the ion exchange resin is characterized as being nitrate enriched.

Typically, the brine exiting the SBR is substantially nitrate-free.

In exemplary embodiments, the inorganic salt is sodium chloride.

In some embodiments, the concentration of the salt entering the column ranges from e.g., 5,000 mg per liter of the brine solution to about e.g., 50,000 mg per liter of the brine. In some embodiments, the concentration of the salt ranges from e.g., 10,000 mg per liter of the brine to about e.g., 20,000 mg per liter of the brine. In some embodiments, the concentration of the salt ranges from e.g., 5,000 mg per liter of the brine to about e.g., 15,000 mg per liter of the brine. In exemplary embodiments, the concentration of sodium chloride in the brine solution is about 25,000 mg per liter of the brine.

Ion exchange is a water treatment system known in the art and can be scaled to fit any size treatment facility. As known in the art, ion exchange resin may be utilized to replace unwanted ions e.g., toxic ions such as nitrate, nitrite, lead, mercury, arsenic and many others, and thus the solution is exchanged for a similarly charged ion attached to an immobile solid particle.

In some embodiments of the present invention, the ion exchange resin is a nitrate selective resin. A nitrate selective resin has higher affinity for nitrate than for other major anions present in the water. In some embodiments, the nitrate selective resin has also high affinity to sulfate.

Ion exchange resin may come, without limitation, in two forms: cation resins, which exchange cations like calcium, magnesium, and radium, and anion resins, used to remove anions like nitrate, arsenate, arsenite, or chromate. Both are usually regenerated with a salt solution e.g., sodium chloride. In the case of cation resins, the sodium ion displaces the cation from the exchange site; and in the case of anion resins, the chloride ion displaces the anion from the exchange site.

As used herein, the term “ion exchange resin” may also encompass mixture of ion exchange resins, or a material made from or comprising at least one ion exchange resin.

As used herein, the term “column” means a vessel or container having at least one opening, and preferably having two openings. Such a vessel or container can be of any shape or size. Thus, as used herein, the term “column” encompasses, for example, tubes, flasks, and reactors of any size and shape, including, but not limited to, small and even microscopic vessels and containers such as, but not limited to, pipette tips.

As used herein, the term “ion exchange column” or “column of an ion exchange resin” means a column that contains an ion exchange material. Exemplary configurations of ion exchange columns are cylinders having openings at opposing ends.

Sequencing Batch Reactor (SBR) systems are known in the art, and has been utilized extensively for carbonaceous, nitrogen and phosphorous removal.

In some embodiments, the pH in the SBR may be adjusted to the range of 7 to 14. In some embodiments, the pH in the SBR may be adjusted to the range of 7 to 10. In exemplary embodiments, the pH of the SBR is kept at a value that ranges from about 7 to about 9. In some embodiments, pH is adjusted to, or kept in, the desired range of about 8.

Typically, but not exclusively, while entering the SBR, the brine solution is characterized by a Cl⁻ concentration of e.g., about 3000 mg/L, 4000 mg/L, 5000 mg/L, 6000 mg/L, 7000 mg/L, 8000 mg/L, 9000 mg/L, 10,000 mg/L, 11,000 mg/L, 12,000 mg/L, 13,000 mg/L, 14,000 mg/L, 15,000 mg/L, 16,000 mg/L, 17,000 mg/L, 18,000 mg/L, 19,000 mg/L, 20,000 mg/L, 21,000 mg/L, 22,000 mg/L, 23,000 mg/L, 24,000 mg/L, 25,000 mg/L, 26,000 mg/L, 27,000 mg/L, 28,000 mg/L, 29,000 mg/L, 30,000 mg/L, 31,000 mg/L, 32,000 mg/L, 33,000 mg/L, 34,000 mg/L, 35,000 mg/L, 36,000 mg/L, 37,000 mg/L, 38,000 mg/L, 39,000 mg/L, 40,000 mg/L, 41,000 mg/L, 42,000 mg/L, 43,000 mg/L, 44,000 mg/L, 45,000 mg/L, 46,000 mg/L, 47,000 mg/L, 48,000 mg/L, 49,000 mg/L, or 50,000 mg/L, including any value therebetween.

In some embodiments, the denitrification is carried out using an electron donor. The term “electron donor” refers to a reducing agent. The terms “reducing agent”, or “reduction agent”, refer to a material, which reacts with a second material and causes the second material to gain electron(s) and/or decreases the oxidation state of the second material. Exemplary electron donors include, but are not limited to, methane, alcohols (e.g., methanol, ethanol), thiols, vinyl ethers, acetic acid, hydrogen gas, and compounds containing carbon to carbon double bonds attached to an aromatic ring.

In exemplary embodiments, the electron donor is ethanol, dosed with KH₂PO₄.

In some embodiments, the pH is adjusted to, or kept in, the desired value using an acid. In some embodiments, the acid is a hydrochloride acid (HCl).

In some embodiments, SBR further comprises denitrifying bacteria. The term “denitrifying bacteria” refers to any bacteria capable of denitrification. Typically, but not exclusively, the denitrification process is outlined according to the following equation:

2NO₃⁻+10e⁻+12H⁺→N₂+6H₂O

In some embodiments, the denitrifying bacterial biomass exiting the SBR is characterized by VSS/TSS value of about e.g., 50%, 55%, 60%, 65%, 70%, 75%, 80%, 85%, including any value therebetween.

Herein, the term “VSS” (total volatile suspended solids) is a measure of suspended solids in the SBR which are volatile. The term “TSS” (Total Suspended Solids) is the total amount of suspended solids in the SBR.

According to some embodiments, the system further comprises oxidation reduction potential (ORP) meter for determining ORP of brine solution inside the sequential batch reactor.

In some embodiments, the ORP value of the brine solution is monitored. In some embodiments, the ORP value of the brine solution is monitored in the SBR. In some embodiments, the ORP value of the brine solution is monitored during the ozonation.

The term “ORP (value)” as used herein and in the art means the unit of oxidation-reduction potential. More specifically, if a certain substance has an ORP value of not more than 0 mV, the substance would be believed to have a reducing power and on the other hand, if the ORP value thereof is not less than 0 mV, the substance would be believed to have an oxidative power. These can be determined using any commercially available measuring machine (e.g., ORP-meter).

ORP may be set at a defined non-negative value, e.g., +500 mV, +400 mV, +300 mV, +200 mV, +100 mV, 0 mV, including any value and range therebetween. ORP may be set at a defined negative value, e.g., −1 mV, −100 mV, −200 mV, −300 mV, −400 mV, −500 mV, including any value and range therebetween.

ORP may be set at a defined negative range of values e.g., −150 mV to −250 mV.

As described in the Example section that follows, multiple electron donor aliquot may be added at a specified intervals using ORP measurement of the batch reactor so as to allow minimizing electron donor addition and dissolved organic-based component, to thereby avoid undesired sulfate reduction.

It is therefore to be recognized that the ORP is used so as to assist to minimize DOC in the system, minimize electron donor addition during denitrification, and/or minimize the amount of ozone necessary to treat SBR effluent (e.g., remove turbidity, disinfect, etc.), thereby avoiding the need to replace the brine and minimizing brine production, hence allowing long term proper operation of the system as disclosed herein.

In some embodiments, the DOC in the recycled regenerant is kept at a low level of e.g., 10 mg/L, 20 mg/L, 30 mg/L, 40 mg/L, 50 mg/L, 60 mg/L, 70 mg/L, 80 mg/L, 90 mg/L, or 100 mg/L, including any value and range therebetween. In some embodiments, the DOC in the recycled regenerant varies within a range of e.g., less than ±5%, less than ±10%, or less than ±20%.

By “avoiding the need to replace the brine and minimizing brine production” it is meant that when the regeneration cycle of the brine biogeneration circuit, as further described below, is performed repeatedly, that is, at least e.g., 20%, 30%, 40%, 50%, 60%, 65%, 70% 75%, or 80% (wt.) of the brine solution is recirculated.

Typically, but not exclusively, the ethanol to nitrate mass ratio is monitored to about 1.68.

Typically, but not exclusively, the nitrate removal rate has a value (in gr N/L_{reactor}/day) that ranges from about e.g., 1 to 5, 2 to 4, 1.5 to 3, including any value and range therebetween.

In some embodiments, the nitrate removal capacity is about e.g., 1 g N/L resin, 2 g N/L resin, 3 g N/L resin, 4 g N/L resin, 5 g N/L resin, 6 g N/L resin, 7 g N/L resin, 8 g N/L resin, 9 g N/L resin, 10 g N/L resin, 11 g N/L resin, 12 g N/L resin, 13 g N/L resin, 14 g N/L resin, or 15 g N/L resin, including any value and range therebetween.

In some embodiments, the nitrate removal capacity varies within a range of e.g., less than ±20%, or less than ±10%, at a defined service length (e.g., of 380 BV).

Typically, denitrified regenerant from the SBR is characterized by turbidity (e.g., about 10 to 20 NTU), derived from e.g., suspended solids, dissolved organic carbon (DOC), e.g., biomass and bacterial contamination in the regenerant.

The term “turbidity” means the cloudiness or haziness of a fluid caused by individual particles (suspended solids). The turbidity can be measured by using Formazin Turbidity Standard and characterized by Nephelometric Turbidity Units (NTU).

It will be appreciated that the nitrate-reduced brine of the present invention may be further treated in order to remove additional impurities. Thus, for example, the present disclosure contemplates an ozonation step of the nitrate-reduced brine following the denitrification process of the present invention to remove any suspended solid contents such as, without limitation, excess biomass, reduce turbidity and disinfect recycled brine.

In some embodiments, an ozonation step is performed so as to reduce the turbidity to a value that ranges from about e.g., 1 NTU to 8 NTU, 2 NTU to 7 NTU, 2 NTU to 6 NTU, 1 NTU to 5 NTU, or 1 NTU to 3 NTU.

As used herein, the term “ozonation” means treating a liquid with ozone. Typically, the ozonation of a liquid is carried out with the aim to reduce the amount of organic compounds present in the liquid and to remove them completely in an ideal case. Typically, but not exclusively, the amount of the ozone used to reduce the turbidity of the regenerant is about 3 to 5 mg O₃ per liter brine.

In some embodiments, the ozonation allows keeping DOC in the recycled regenerant at a low level, as described hereinbelow (e.g., about 60 mg/L).

As described hereinbelow and without being bound by any particular theory, during ozonation the suspended solids forming the turbidity are concentrated as foam that constituted e.g., about 0.1 to 0.5% of the treated brine on a mass basis. Such amounts can be easily eliminated through evaporation.

In some embodiments, the ozonation allows to increase the biodegradability of the SBR effluent by at least 1%, 5%, 10%, 15%, 20%, 25%, 30%, 40%, or 50%, including any value and range therebetween. In some embodiments, the ozonation allows to increase the biodegradability of the SBR effluent by 20% to 30%. As described hereinabove, the ozonation step may further comprise ORP measurement of the ozonation unit to control the amount of the ozone added to the brine and flow of brine from ozonation unit to the disclosed ion exchange unit.

In some embodiments, at the end of the ion exchange unit regeneration (i.e., when the ion exchange column is filled with brine solution) the ion exchange column is further filled with disinfectant solution at the upper side of the column.

In some embodiments, the ion exchange column is filled with disinfectant solution after air purging of the column so as to substantially remove remaining brine solution from the column.

Non-limiting exemplary disinfectants are types of peroxygen (peroxide, peracid, combination of peroxide/peracid, etc.). In exemplary embodiments, the disinfectant is H₂O₂ solution (e.g., 0.2% (wt.).

In some embodiments, following the filling of the ion exchange column with disinfectant solution, the disinfectant is discharged from the column. In some embodiments, the disinfectant is discharged from the bottom of the column. In some embodiments, the disinfectant is discharged at low flow regime, as described below, so as to minimize the waste brine volume.

General:

As used herein the terms “approximately” and “about” which are used hereinthroughout interchangeably refer to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, biological, and biochemical arts.

As used herein, the term “treating” includes abrogating, substantially inhibiting, slowing or reversing the progression of a condition, substantially preventing the appearance of an undesired condition.

In addition, where there are inconsistencies between this application and any document incorporated by reference, it is hereby intended that the present application controls.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples which, together with the above descriptions, illustrate the invention in a non-limiting fashion.

### Example 1

### Material and Methods

Experimental System:

The experimental system comprises three main elements: (1) ion exchange (IX) columns for nitrate removal from the feed water, (2) denitrification sequential batch reactor (SBR) for nitrate elimination from the brine, and (3) ozonation for post treatment of the SBR's brine effluent. The setup of the system is further discussed hereinabove and illustrated in FIGS. 1A-C. In an exemplary configuration, the entire system was controlled by a programmable logic controller (Vision 570, Unitronix, Airport City, Israel).

Ion Exchange:

Three 8 L columns were first filled with 1 L of basalt gravel (2-5 mm) for even drainage and on top 5 L (or 1 bed volume, BV) of a nitrate selective resin (A-520E, Purolite, Bala Cynwyd, Pa.). The columns were operated through two basic cycles: the service cycle (nitrate adsorption from the water supply) and the regeneration cycle that restores resin capacity after exhaustion. The regeneration cycle consisted of three steps: regeneration (brining), disinfection, and rinse followed by a standby mode. At all times one column was in service cycle, another in regeneration cycle and another in standby mode. Feed water and rinsing flow rates were maintained at 100 L/h, while regeneration (18 BV) and disinfection (2 BV) flow rates were maintained at 18 L/h to comply with resin manufacturer recommendations. Feed water was composed of tap water mixed with an artificial nitrate solution at a final concentration of 25±1 mg N/L. Detailed influent water characteristics are given in Table 1 below which presents the effect of ion exchange service cycle length on the concentration of nitrate and sulfate removed, chloride added and the Cl/N equivalent ratio for the various adsorption cycle bed volumes. Feed water NO₃⁻—N, SO₄⁻²—S, and Cl⁻ concentrations (mg/L) were 25.9±0.6, 16.2±0.3 and 247±4, respectively. Hereinthroughout, symbols such as: “NO₃—N”, “SO₄⁻²S” refer to the corresponding mass of N, and S, respectively, derived from the corresponding ions.

TABLE 1

| Bed Volumes | NO₃-N removed mg/L | SO₄-S removed mg/L | Cl⁻ added mg/L | $\frac{eq . {Cl}^{-} added}{eq . N removed}$ |
|---|---|---|---|---|
| 160 | 24.9 ± 0.3 | 11.8 ± 0.4 | 100.9 ± 6.5 | 1.54 |
| 320 | 18.8 ± 0.8 | 5.6 ± 0.3 | 68.0 ± 4.3 | 1.42 |
| 380 | 16.7 ± 0.9 | 3.8 ± 0.3 | 46.9 ± 2.3 | 1.11 |
| 480 | 13.6 ± 0.9 | 2.8 ± 0.5 | 36.4 ± 9.4 | 1.06 |

In exemplary procedures, four service lengths were examined: 160, 320, 380, and 480 BV. Operation under each mode was carried out over at least 40 consecutive cycles. An automatic sampler (Sigma SD900, Hach, Loveland, Colo.), set to operate hourly during the service cycle, was used to prepare individual and composite samples. Chloride concentration in the brine was maintained at 14-16 g/L (2.5% NaCl solution) by the addition of a 9% NaCl solution. This relatively low salt concentration was chosen to facilitate biological activity in the denitrification unit and to allow for smaller brine wastewater production during rinsing the resin with freshwater after the regeneration stage. Disinfection was carried out using 0.2% H₂O₂ solution. All solutions for the regenerant were prepared using softened tap water.

SBR:

In exemplary procedures, denitrification was carried out in a cylindrical container (130 L, 40 cm ID) using ethanol (EtOH) as the electron donor. The EtOH was dosed together with 0.5 g KH₂PO₄ per batch. Sludge was not intentionally removed during the experimental period and minimal periodic mixing (50-60 rpm) was maintained. pH was controlled to pH 8.2 by adding 6% solution of HCl during SBR mixing (Alpha 190 pH/ORP controller, Eutech Instruments Pte Ltd, Singapore and an epoxy pH electrode, Van London Co., Houston Tex.).

Ozonator:

In exemplary procedures, ozone was produced by passing O₂ (Nuvo Lite 920 oxygen concentrator, Nidek Medical Products, Birmingham, Ala.) through a 4 g O₃/h ozone generator (CD 10, ClearWater Tech, LLC., San Luis Obispo Calif.). Using a Venturi tube, ozone was injected into the recirculating stream of a 40 L (160 mm diameter, 200 cm height) transparent PVC contact column. The retention time was 40-50 min and the ozone dosage was 2-5 mg O₃/L. Output of the ozone generator was controlled by oxidation reduction potential measurement (Alpha 190 pH/ORP controller, Eutech Instruments Pte Ltd, Singapore and industrial ORP electrode, Cole Parmer Instruments Company, Vernon Hills, Ill.).

Analyses:

In exemplary procedures, nitrate, nitrite, chloride and sulfate concentrations were determined by ion chromatography (761 Metrohm ion chromatograph equipped with 150 mm MetrosepA Supp5 column and precolumn, Metrohm AG, Herisau Switzerland) using an eluent containing 3.2 mM Na₂CO₃ and 1.0 mM NaHCO₃. Total Organic Carbon (TOC) concentration was determined by a TOC-VCPH analyzer (Shimadzu, Kyoto, Japan). DOC concentration was determined by performing TOC analysis on samples filtered through 0.22 μm syringe filter. Turbidity was determined using a Hach 2100Q turbidometer (Loveland, Colo.). Heterotrophic plate count (HPC) was performed according to the spread plate method (APHA, 1995).

### Example 2

### Determination of Optimal Length of the Ion Exchange (IX) Service Cycle

Without being bound by any particular theory, the choice of the length of the IX service cycle, when nitrate in the feed water is adsorbed onto the resin, has implications on the process. The duration of the service cycle affects the chemical composition of the product water, particularly the chloride concentration, the amount of waste brine generated per volume of final product water, and the considerations whether to treat all (“full treatment”) or part of the groundwater (“split treatment”) as will be explained below.

The appearance of nitrate breakthrough in the nitrate selective resin used (Purolite A520E) is normally observed at short service cycle lengths of 200 to 300 BV (Bae et al., 2002; McAdam et al., 2010). In a “split treatment” scheme, an IX column is operated at short service cycle lengths, and the product water containing minimal nitrate concentrations is mixed with untreated water in order to reduce treatment costs per cubic meter. However, when sulfate concentrations in the feed water are significant (>10 mg/l as S) as is normally the case in natural groundwater, both nitrate and sulfate are exchanged for chloride at short service cycle lengths resulting in high chloride concentrations in the product water. The adsorption of sulfates also leads to sulfate buildup in the recirculating brine, reaching concentrations of several g/L brine depending on amount of regenerant blow down (Bae et al., 2002; Clifford and Liu, 1993; van der Hoek et al., 1988).

In the case of sulfate, breakthrough occurs in A520E earlier than nitrate, at around 100 BV and ends at 300 BV when the sulfate concentration in the product water approaches that of the feed water (Bae et al., 2002). During this stage, adsorbed sulfate is also released back to the treated water as sulfate is exchanged with the more favorable nitrate. This phenomenon is sometimes referred to as “sulfate dumping” (DeSilva, 2010), because the sulfate concentration in the treated water is observed to exceed that of the feed water at this stage. Allowing full sulfate dumping to occur by adequately increasing the service length reduces chloride addition to product water and lessens sulfate buildup in the regenerant, but at a price of higher nitrate concentration in the product water.

In order to assess the optimal duration of the service cycle and to determine whether to operate the process using a ‘split treatment’ or ‘full treatment’ scheme, four run lengths were tested: 160 BV, 320 BV, 380 BV and 480 BV. Table 1 above shows the effect of service length on chloride addition to the product water with respect to removed nitrate and sulfate.

Shorter service cycle lengths resulted in much higher amounts of chloride added with a Cl⁻/NO₃⁻—N equivalent exchange ratio of 1.54 at 160 BV (Table 1). Longer service cycle lengths resulted in lower amounts of chloride added to the product water and nitrate removed with the Cl⁻/NO₃⁻—N equivalent exchange ratio dropping to 1.06 at 480 BV. Without being bound by any particular mechanism, this result is attributed to sulfate adsorption and dumping as well as to nitrate breakthrough. The increase in nitrate concentration in the product water with increasing service length finally exceeded the regulation limit of 10 mg NO₃⁻—N/L (EPA, 2009) in the 480 BV test case. It should be noted that the feed water contained a relatively high concentration of chlorides (around 250 mg/L) not characteristic to groundwater, and the results are expected to improve at lower chloride concentrations due to better adsorption of nitrate on the resin.

Water from a short 160 BV IX service cycle can be mixed with untreated water in a ‘split treatment’ scheme. In this case, approximately 68% of the water would be treated by IX and the remaining 32% untreated water would be blended. The resulting water would have a the same nitrate concentration as for a treatment of all the water from a given well using a service cycle of 380 BV (i.e. “full treatment”), but the addition to the chloride concentration would be higher, 65 mg/L versus 47 mg/L, respectively.

Another advantage associated with longer service cycle length is that less service cycles are necessary to achieve a given volume of product water and less waste brine is produced. This is because at the end of each regeneration cycle, a certain amount of waste brine is inevitably produced and must be discarded when the regenerated resin is washed with fresh water before column reuse. In the present disclosure, 1.6 more service cycles were needed for the ‘split treatment’ scheme as opposed to ‘full treatment’ of the entire water volume.

Based on the above results, the optimal service length was determined to be 380 BV for the given feed water composition and that the entire flow of feed water should be treated instead of a ‘split treatment’ scheme. Under lower concentrations of nitrate, chloride and sulfate in the feed water, it is expected that the length of the service cycle would increase together with improvement of product water quality and minimization of waste brine.

### Example 3

### System's Performance

In exemplary procedures, the system's performance was tested during operation at a service cycle length of 380 BV.

Following the aforementioned evaluation, the system was operated at a service length of 380 BV for about a year. During this time the nitrate removal capacity decreased only slightly: from 6.0±0.9 to 5.7±0.2 g N/L resin. Average product water NO₃⁻—N, SO₄⁻²—S and Cl⁻ concentrations (mg/L) were 9.2±0.6, 12.4±0.3, 294±9, respectively, showing that nitrate was removed to the maximal allowable concentration. Composite samples showed nearly no change in product water alkalinity (132±9 mg/L as CaCO₃) as compared to the feed water (137±12 mg/L as CaCO₃). The lower sulfate concentration in the product water, as compared to the feed water, indicated that sulfate was removed from the feed water and concentrated in the brine. This was corroborated with measured sulfate levels of 1.7 g SO₄⁻²—S/L in the recirculating brine at steady state. DOC concentrations in the product water were 2.1±0.8 mg/L and on average 0.5 mg/L lower than the feed water (2.6±0.6 mg/L). This measurement was attributed, without being bound by any particular mechanism, to DOC adsorption on the resin during the service mode because IX resins are known to adsorb organic micropollutants such as aromatic compounds, chlorinated solvents, herbicides and nitrosamines from drinking water and because DOC adsorption on the tested chloride-saturated resin was reported to be appreciable.

It is noteworthy that these long term steady state results of lower product water DOC suggest that DOC in the recirculated regenerant does not accumulate on the resin and released to product water. The heterotrophic plate count in the product water was maintained at acceptable levels of 10-700 CFU/mL (CFU: colony-forming units) before final disinfection and optimization of the resin disinfection procedure.

### Example 4

### Column Regeneration and Waste Brine Production Per Cycle

In exemplary procedures, after completion of the service cycle, the exhausted IX column was regenerated with using 18 BV of a brine solution containing an average concentration of 15,185±622 mg/L Cl⁻. Measures were taken during the IX column's transition from potable water to brine and back again in order to minimize waste brine production. This included forced air displacement of the liquid volume in the IX column at the end of the service cycle and at the end of the regeneration cycle when the column was full of brine. In addition, it was found that a slow rate of filling and discharge of H₂O₂ disinfectant following brine purge at the end of the regeneration cycle reduced the volume of waste brine generated.

Table 2 below shows parameters of waste production from the process divided into four different streams: 1) brine waste or excess regenerant from adding ethanol, makeup NaCl and HCl, 2) brine waste from initial disinfection of the column, 3) wastewater from second phase of disinfection and 4) wastewater from final column flush.

TABLE 2

| | Regenerant | Disinfection | Disinfection | |
|---|---|---|---|---|
| Parameter | waste | waste 1 | waste 2 | column flush |
| BV | 0.28 ± 0.15 | 0.66 ± 0.11 | 0.89 ± 0.16 | 0.96 ± 0.15 |
| Cl⁻ mg/L | 13,950 ± 560 | 6,060 ± 210 | 377 ± 22 | 362 ± 18 |
| EC (mS/cm) | 57 ± 1.0 | 27.7 ± 1.5 | 1.4 ± 0.1 | 1.1 ± 0.1 |
| Discharge to | truck | truck | sewage | sewage |

Operating the combined system with a service cycle duration of 380 BV resulted in the production of 0.94 BV of waste brine per cycle (brine waste 1 and 2) which corresponds to only 0.25% of the treated water volume. Although only a small amount of waste brine was produced per cycle, it could not be discharged to sewage due to high electrical conductively (EC) and requires costlier removal by truck.

IX disinfection and flush water (wastewater 3 and 4) resulted in the production of an additional 1.85 BV of wastewater, however with an EC low enough to be discharged to sewage. The mass of wasted NaCl discharged per volume of product water was very low, 35.7 kg NaCl/1000 m³, 34% less than a similar IX bioregeneration process and is 10 fold less when compared to conventional ion exchange (380 kg NaCl/1000 m³ product water) (Clifford, D. and Liu, X. S., Journal American Water Works Association 85 (4), 135-143. 1993). Although the brine waste volumes on a full scale operation are thus expected to be manageable, low process blow down may act negatively on the composition and on the quality of the recycled regenerant and deter long term sustainable service cycle operation. Including the salt lost during regeneration and column disinfection, the combined process required total of 1.62 equivalents of Cl⁻ to every 1 equivalent of NO₃⁻—N removed.

### Example 5

### Regenerant Denitrification in SBR

The characteristic fluctuations in nitrate concentration found in the regenerant brine during column regeneration made application of a continuous denitrifying reactor problematic, thus a sequential batch reactor (SBR) was employed. Exhausted regenerant was collected batch-wise containing a nitrate load of about 30 g NO₃⁻—N per cycle with an average concentration of 317±25 mg/L. In addition to sulfate buildup in the recirculating brine as hereinabove mentioned, alkalinity was also increased due to denitrification, to 6950±60 mg/L as CaCO₃. These concentrations did not affect denitrification or the amount of nitrate exchanged per cycle. Depending on the length of the service cycle, the SBR was operated at an 8, 16 or 24 hr interval. The sludge that developed in the SBR was granular in nature and had marked settling properties with a sludge volume index (SVI) of 15.7±2.6 ml/g and VSS/TSS of 79.4±3.4%. The sludge volume in the SBR, after settling, was constant at 18-20 L and excess sludge was not intentionally removed. The only sludge that washed out of the system was due to residual flocs in the piping (about 40 mL/batch after settling) and such small amounts of sludge can be eliminated by simple evaporation.

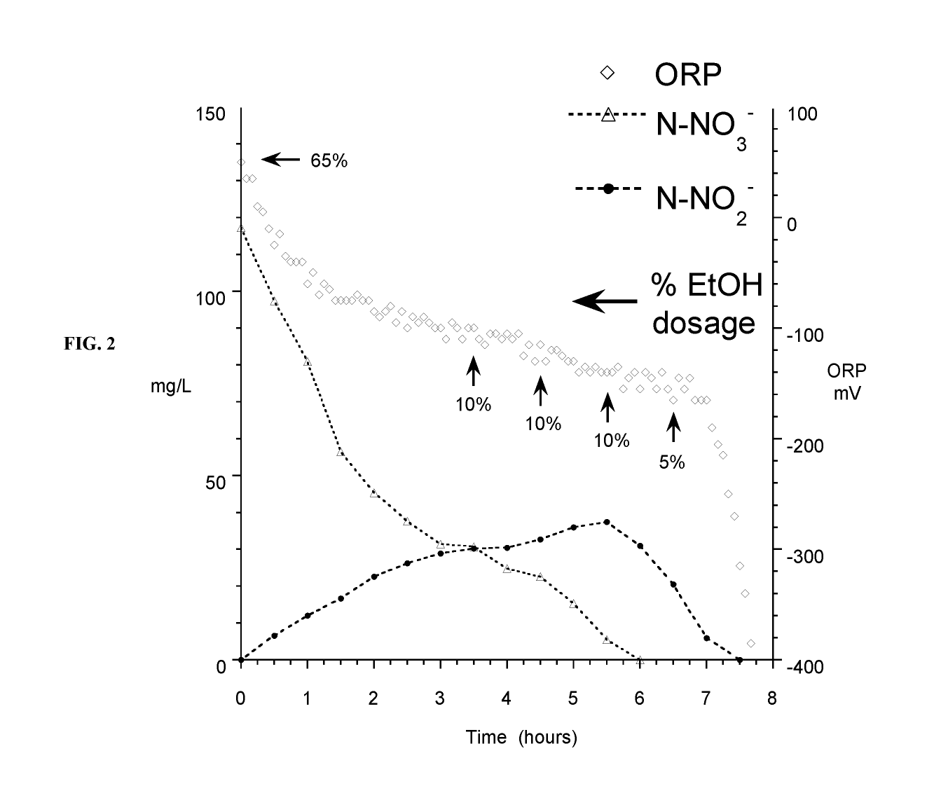

Earlier experiments with a 12 L bench scale SBR demonstrated the ability to monitor the denitrification process by ORP measurements and control the addition of ethanol to ensure complete denitrification without overdosing. ORP, NO₃⁻ and NO₂⁻ concentrations during denitrification are depicted in FIG. 2, showing that the ORP values drop upon completion of nitrate and nitrite. Rather than giving ethanol in one large dose at reactor filling, a strategy was developed to divide the ethanol dose into smaller amounts and give them at predetermined time intervals as long as the SBR maintained an ORP above a given set point (e.g., −200 mV). This was largely carried out to limit excess ethanol concentrations in the SBR that may encourage sulfate reduction. Higher ORP levels were maintained in the SBR when using the stepwise method of ethanol feeding making for better monitoring of denitrification. Further improvements to ethanol usage were achieved in the 130 L SBR by delaying the initial ethanol dose for a short period of time to allow for consumption of residual organics and sulfides that may accumulate at the end of the previous batch.

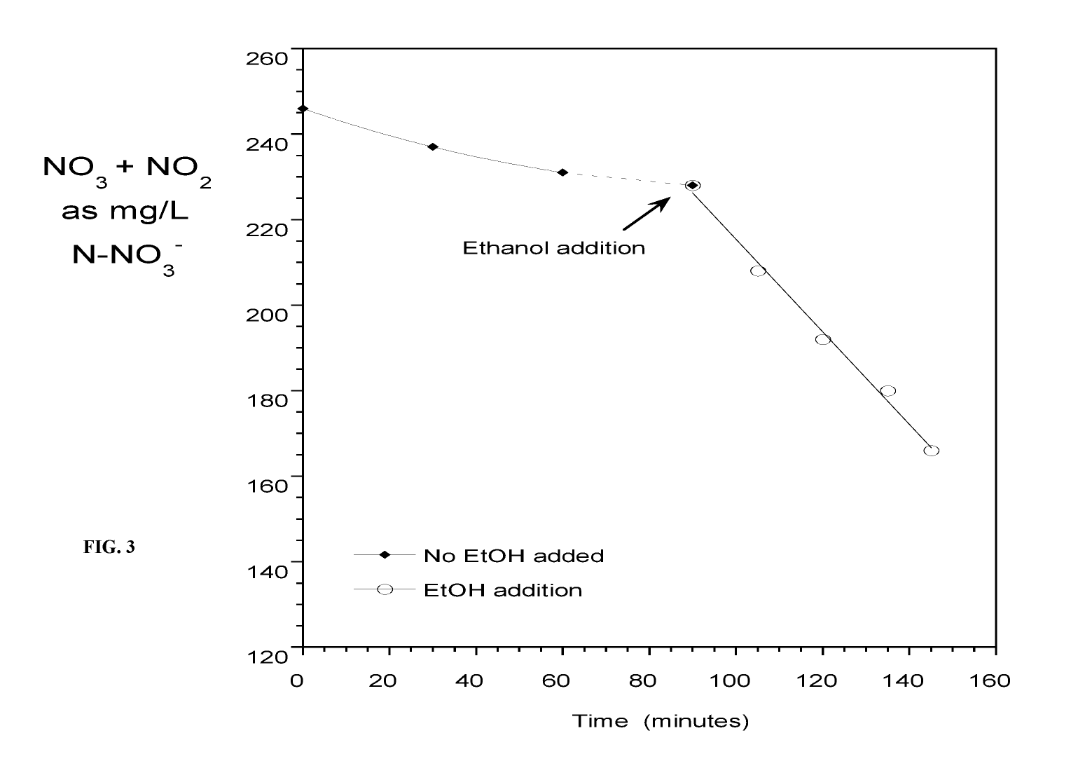

FIG. 3 shows a significant denitrification rate in the SBR during the initial 90 minute delay without ethanol addition, about 20% of the denitrification rate when ethanol was added stepwise.

The SBR unit typically achieved complete nitrate removal with nitrate removal rates of 2.6±0.4 g N/L_{reactor}/d and an ethanol to N-NO₃⁻ nitrate mass ratio of 1.68±0.18. The low ethanol to N-NO₃⁻ ratio is reflected in long sludge age calculated to be about 500 days, demonstrating that most of the electrons contained in ethanol were going to catabolism.

### Example 6

### Polishing of the Denitrified Regenerant by Ozonation

Typically, denitrified regenerant from the SBR contains suspended solids, DOC and bacterial contamination that must be removed to ensure a prolonged reuse and prevent resin fouling.

Typical SBR effluent turbidity and suspended solids values in the present disclosure were 16.7±6.4 NTU and between 20 to 40 mg/L. Simple filtration in sand or GAC columns as well as coagulation/flocculation did not effectively reduce the turbidity or tendency to clog during filtration. In order to reduce resin biofouling and bacterial contamination, the initial polishing treatment selected was an aerobic membrane bioreactor (MBR). While turbidity and bacterial counts of the MBR's effluent were low, regenerant DOC values were high (152±6 mg/L), which promoted biofilm growth on the pipes and bacterial contamination in the product water.

In exemplary procedures, disinfection of the piping and the IX columns using H₂O₂ was conducted at the end of the regeneration cycle. Moreover, the sludge in the MBR had very poor settling characteristics due to the low food to microorganism ratio (F/M) and resulted in membrane fouling. However, efforts to reduce sludge age in the MBR resulted in an unaffordable waste brine volume. As a result of the aforementioned reasons, the MBR was abandoned in favor of ozonation, used for the first time in such a process.

In exemplary procedures, ozonation was found to highly reduce turbidity to 2.8±1.0 NTU and enhance filterability. These values were similar to the values measured in the MBR effluent. During ozonation, the suspended solids forming the turbidity were concentrated as foam that constituted about 0.3% of the treated brine on a mass basis. Such amounts can be eliminated through evaporation. Although foam was formed even when air was bubbled through the regenerant, the presence of ozone was proved to be critical in attaining satisfactory turbidity levels and maintaining high filterability.

Typical ozone demand was about 3 to 5 mg O₃ L/brine. However, when ethanol was significantly over or under dosed during denitrification due to malfunction or inadequate control, ozone demand increased up to ten fold in order to oxidize residual nitrite or sulfide concentrations. In addition, ozonation may cause the formation of bromate, however, none were detected in the product water.

In spite of the system's low brine blow down, DOC in the recycled regenerant after more than a year of continuous operation was maintained at relatively lower levels of 61±11 mg/L suggesting that ozonation breaks down a significant of the residual organic compounds originating from biological denitrification. Based on oxygen uptake tests, ozonation was estimated to increase the biodegradability of the SBR effluent by approximately 28% (results not shown). As mentioned hereinabove, the remaining DOC did not interfere with IX resin exchange capacity, however, it was necessary to maintain a stringent disinfection program to prevent bacterial regrowth and contamination throughout the system.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method comprising the steps of:
(a) contacting a nitrate contaminated water with one or more columns of ion exchange resins having an affinity to nitrate, thereby removing nitrate from the water and loading nitrate in said one or more columns of said exchange resins;
(b) separating said reduced nitrate content product water from the nitrate loaded columns of said ion exchange resin, thereby forming a product water having reduced nitrate content;
(c) forming a regenerated ion exchange resin having reduced nitrate load, comprising the steps of:
(i) contacting the nitrate loaded columns of said ion exchange resin with a fed brine solution having nitrate desorbing content; and
(ii) removing the brine solution from the treated ion exchange resin, thereby forming a regenerated ion exchange resin having reduced nitrate load;
(d) contacting the brine solution to a sequential batch reactor (SBR) comprising denitrifying bacteria;
(e) adding an electron donor to the SBR thereby essentially removing nitrate from the brine solution;
(f) performing sedimentation of the brine solution and adding salt thereto to thereby remove excess denitrifying bacterial biomass therefrom; and
(g) contacting the brine solution with O₃ thereby disinfecting and/or removing remaining suspended solids, turbidity and dissolved organic-based component in said brine and optionally recycling the brine to step (c), thereby forming a regenerated ion exchange resin having reduced nitrate load.

2. The method of claim 1, characterized by one or more from (i) to (v):
(i) steps (a) to (b) and/or (c) to (g) being performed repeatedly;
(ii) being performed such that at least 75% (wt.) of the brine solution present in step (d) is recycled to step (c) following step (g);
(iii) step (b) being recycled to step (a);
(iv) at least one of steps (a) and (b), and at least one of steps (c) to (g) being performed simultaneously;
(v) at least one of steps (a) and (b), and at least one of steps (c) to (g) being performed in a different column of ion exchange resin.

3. (canceled)

4. (canceled)

5. (canceled)

6. (canceled)

7. (canceled)

8. The method of claim 1, further comprising one or more from steps (i) to (iv):
(i) performing oxidation reduction potential (ORP) measurement of said SBR to control electron donor addition;
(ii) performing ORP measurement of said ozonation unit thereby controlling ozone addition and/or a flow of brine from ozonation unit to ion exchange unit;
(iii) keeping pH of said SBR at a value that ranges from about pH 7 to about 9
(iv) discharging the brine in said SBR.

9. The method of claim 8, wherein said electron donor addition is performed in aliquots at a specified time interval, thereby minimizing electron donor addition and dissolved organic-based component.

10. (canceled)

11. The method of claim 1, wherein said desorbing content of step (c) comprises chloride anions in concentration of at least 10,000 mg/L.

12. The method of claim 1, wherein said dissolved organic-based component is or derived from denitrifying biomass and/or bacterial component.

13. (canceled)

14. A system comprising:
an ion exchange unit comprising at least one column of an ion exchange resin;
a brine bioregeneration circuit comprising an SBR; and
an ozonation unit,
wherein said ion exchange unit, said SBR, and ozonation unit are in fluid communication with each other.

15. The system of claim 14, wherein said ion exchange unit comprises at least two columns of an ion exchange resin.

16. The system of claim 14, further comprising a settling tank, being in fluid communication to said ozonation unit and to said SBR, optionally, wherein said fluid is a brine solution.

17. (canceled)

18. The system of claim 16, characterized by one or more of (i) to (iii):
(i) said SBR of the brine bioregeneration circuit being disposed downstream of said ion exchange unit and is configured to receive the brine solution from at least one ion exchange column;
(ii) said settling tank being disposed downstream of said SBR and is configured to receive said fluid from said SBR;
(iii) said ozonation unit being disposed downstream of said settling tank and is configured to receive the fluid from said settling tank.

19. (canceled)

20. (canceled)

21. The system of claim 14, further comprising a pipe attached to, or integrally formed with said ozonation unit, wherein said pipe is configured to lead a brine solution out of said ozonation unit and enter the ion exchange unit, optionally, wherein said ion exchange resin is a nitrate selective resin.

22. (canceled)

23. The system of claim 14, wherein said ion exchange unit comprises:
(a) a first water inlet configured to provide nitrate contaminated water to said at least one column of said ion exchange resin;
(b) a second water inlet configured to provide a brine solution from said ozonation unit to said at least one column of said ion exchange resin;
(c) a first water outlet configured to allow an exit of nitrate-reduced water from said least one ion column of said ion exchange resin; and
(d) a second water outlet configured to transfer the brine solution from said at least one column of said ion exchange resin to said sequential batch reactor

24. The system of claim 17, characterized by one or more from:
(i) the brine solution comprising chloride anions at a concentration that ranges from about 10,000 milligrams per liter (mg/L) to about 50,000 mg/L;
(ii) the nitrate-reduced water comprises nitrate in concentration of less than 15 mg/L;
(iii) the nitrate-reduced water comprises chloride anions at a concentration of less than 430 mg/L.

25. (canceled)

26. (canceled)

27. The system of claim 14, further comprising one or more from:
(i) a pH meter configured to determine a pH of a fluid inside said SBR;
(ii) an ORP meter configured to determine ORP of a brine solution inside said sequential batch reactor.

28. (canceled)

29. The system of claim 14, further comprising a pipe attached to, or integrally formed with said SBR, wherein said pipe is configured to lead one or more from (i) to (iii):
(i) an electron donor into said sequential batch reactor;
(ii) an acid into said SBR;
(iii) a salt solution into said SBR.

30. The system of claim 29, wherein said electron donor is one or more materials selected from the group consisting of: acetic acid, ethanol, and hydrogen gas.

31. (canceled)

32. The system of claim 29, wherein said acid is a hydrochloride acid.

33. The system of claim 14, wherein said SBR further comprises denitrifying bacteria.

34. (canceled)

35. The system of claim 29, wherein said salt is sodium chloride.

36. The system of claim 14, further comprising one or more elements from (i) and (ii):
(i) a pipe attached to, or integrally formed with said ion exchange unit, wherein said pipe is configured to lead a disinfectant solution to said ion exchange unit, optionally, wherein said disinfectant solution is hydrogen peroxide;
(ii) a settling tank, being in fluid communication to said ozonation unit and to said SBR and a pipe attached to, or integrally formed with said settling tank is configured to lead salt solution into said settling tank, said salt solution comprising chloride anions.

37. (canceled)
